(19) United States
(12) Patent Application Publication
(10) Pub. No.: US 2006/0122426 A1
(43) Pub. Date: Jun. 8, 2006

(54) Method for producing phthalic acid dichloride

(76) Inventors: Jorn Stolting, Koln (DE); Reinhard Lantzsch, Wuppertal (DE)

Correspondence Address: BAYER CROPSCIENCE LP;Patent Department, 100 BAYER ROAD, PITTSBURGH, PA, 15205-9741, US

(21) Appl. No.: 10/524,748

(22) PCT Filed: Aug. 6, 2003
(86) PCT No.: PCT/EP03/08684

§ 371 Date: Aug. 18, 2005

(30) Foreign Application Priority Data
Aug. 16, 2002 (DE) 102 37 579.8

(51) Int. Cl. C07C 51/58

(52) U.S. Cl. 562/854

(57) ABSTRACT

The invention relates to a novel method for producing phthalic acid dichloride (benzene-1,2-dicarbonylchloride) from phthalic anhydride by reaction with phosgene in the presence of N,N-dialkylformamides.

The invention relates to a novel process for preparing phthaloyl chloride (benzene-1,2-dicarbonylchloride) from phthalic anhydride.

It is already known that phthaloyl chloride can be prepared from phthalic anhydride by reacting with suitable agents for the introduction of chlorine (“chlorinating agents”).

For example, phthaloyl chloride is obtained by reacting phthalic anhydride with trichloromethane or tetrachloromethane in the presence of zinc chloride (cf. U.S. Pat. No. 2,051,096). However, this reaction requires very high temperatures; in addition, trichloromethane and tetrachloromethane are nowadays very problematic reaction components for industrial purposes.

Phthaloyl chloride can also be obtained by reacting phthalic anhydride with thionyl chloride in the presence of zinc chloride (cf. J. Am. Chem. Soc. 1937, 59, 206-208). This reaction too requires very high temperatures. Yield and quality of the product are not entirely satisfactory in this method.

In addition, phthaloyl chloride can also be obtained by reacting phthalic anhydride with phosphorus(V) chloride (phosphorus pentachloride) (cf. Can. J. Chem. 1970, 48, 3566-3571). Here too, the yield of the desired product is very unsatisfactory.

As a further means of preparing phthaloyl chloride, the reaction of phthalic anhydride with trichloromethylisocyanide dichloride in the presence of iron(III) chloride is known (cf. DE-A 20 36 171). However, chlorocarbonylisocyanide dichloride is obtained as a coproduct in this reaction.

Finally, the reaction of phthalic anhydride with phosgene in chlorobenzene in the presence of N,N-dimethylformamide is also known as a means of preparing phthaloyl chloride (cf. U.S. Pat. No. 3,810,940). In this reaction too, the yield of the desired product is not entirely satisfactory.

It is therefore an object of the present invention to provide a process suitable for industrial realization, by which phthaloyl chloride can be obtained in very good yields from a readily available starting material, for example phthalic anhydride, using inexpensive assistants, with acceptable energy demands, and avoiding the occurrence of relatively large amounts of coproducts.

It has now been found that, surprisingly, starting from phthalic anhydride, using phosgene as an agent for the introduction of chlorine and an assistant from the group of the N,N-dialkylformamides and an inert diluent, the desired phthaloyl chloride product can be obtained in high yields and in very good quality when the metering-in of phosgene and N,N-dialkylformamide is not carried out all at once, but rather each component is metered continuously or semicontinuously.

In the context of the invention, continuously means that the particular reaction component (phosgene and/or N,N-dialkylformamide) is metered constantly and uniformly into the reaction mixture over the entire reaction time.

In the context of the invention, semicontinuously means that the particular reaction component (phosgene and/or N,N-dialkylformamide) is metered into the reaction mixture in portions, distributed over defined periods. The individual portions are preferably of equal size and the individual periods are preferably of equal length.

The present invention thus provides a process for preparing phthaloyl chloride of the formula (I) 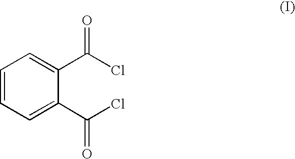
in which phthalic anhydride of the formula (II) 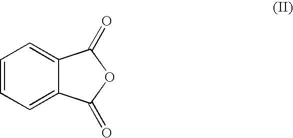
is reacted with phosgene in the presence of an N,N-dialkylformamide of the formula (III) 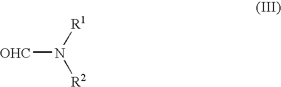
in which

R¹ and R² are each independently straight-chain or branched alkyl,

and in the presence of an inert diluent at temperatures between 20° C. and 150° C.,

characterized in that the amounts of phosgene and of the N,N-dialkylformamide of the formula (III) used are each independently metered in continuously or semicontinuously.

After the reaction has been carried out, the desired product may be obtained in high yield and in very good quality by distillation.

The phthalic anhydride of the formula (II) to be used as starting material in the process according to the invention is a known commercial synthesis chemical.

The phosgene used as an agent for the introduction of chlorine is likewise known.

The process according to the invention is carried out using an N,N-dialkylformamide of the formula (III). In this formula (III), R¹ and R² are each independently preferably straight-chain or branched C1-C₁₀-alkyl, more preferably C₁-C₆-alkyl.

Examples of N,N-dialkylformamides of the formula (III) include: N,N-dimethylformnamide, N,N-diethylformamide, N,N-di-n-propylformamide, N,N-diisopropylformamide, N,N-di-n-butylformamide and N,N-diisobutylformamide.

The N,N-dialkylformamides are known organic synthesis chemicals or reagents.

The process according to the invention is carried out in the presence of an inert diluent. Useful diluents are in particular: hydrocarbons such as pentane, hexane, heptane, octane, cyclohexane, methylcyclohexane, benzene, toluene, xylene, and halogenated hydrocarbons such as dichloromethane, trichloromethane, tetrachloromethane, chlorobenzene or dichlorobenzene. Toluene and chlorobenzene are particularly preferred as diluents.

The process according to the invention may be carried out within a relatively large temperature range. Preference is given to carrying out the reaction at temperatures between 40° C. and 120° C., in particular between 55° C. and 100° C.

The process according to the invention is generally carried out under standard pressure. However, it is also possible to carry out the process according to the invention under elevated or reduced pressure—generally between 0.1 bar and 50 bar, preferably between 1 bar and 10 bar.

To carry out the process according to the invention, for 1 mol of phthalic anhydride of the formula (II), generally between 1.2 mol and 2.5 mol, preferably between 1.4 mol and 2.2 mol, of phosgene, and also between 0.01 mol and 0.20 mol, preferably between 0.02 and 0.10 mol, of N,N-dialkylformamide of the formula (III) are used.

In a preferred embodiment of the process according to the invention, the phthalic anhydride is initially charged in an inert diluent, and the mixture is heated to the reaction temperature.

The phosgene and the N,N-dialkylformamide of the formula (III) are then each metered in continuously, distributed over the entire reaction time, or semicontinuously, i.e. distributed between approximately equally long periods and divided into approximately equally large portions according to the number of these periods.

In a preferred variant, both the phosgene and the N,N-dialkylformamide of the formula (III) are metered in continuously.

In another preferred variant, both the phosgene and the N,N-dialkylformamide of the formula (III) are metered in semicontinuously, distributed between several portions.

In a further preferred variant, the phosgene is metered in continuously, while the N,N-dialkylformamide of the formula (III) is metered in semicontinuously, distributed between several portions.

In a further preferred variant, the phosgene is metered in semicontinuously, distributed between several portions, while the N,N-dialkylformamide of the formula (III) is metered in continuously.

Particularly advantageous in each case is a reaction time of from 5 to 15 hours (depending upon the batch size), in which case the N,N-dialkylformamide of the formula (III) is metered in every 15 to 90 minutes and the phosgene is metered in continuously or semicontinuously.

On completion of the addition of phosgene and N,N-dialkylformamide of the formula (III), the reaction mixture is advantageously kept at the reaction temperature specified for another 1 to 2 hours and subsequently worked up by distillation under reduced pressure.

The inventive preparation of phthaloyl chloride is described in the examples below, which further illustrate the above description. However, the examples should not be interpreted in a restrictive manner.

### PREPARATION EXAMPLES

### Example 1

148 g (1.0 mol) of phthalic anhydride are dissolved 150 ml of toluene and the mixture is heated to 70° C. At this temperature, 0.8 g (5 mmol, total amount: 5.6 g) of N,N-dibutylformamide and 19.8 g (0.2 mol, total amount: 138.5 g) of phosgene are metered in every hour for 6 hours (7 portions in total starting with hour 0). Subsequently, the reaction mixture is stirred at 70° C. for a further 2 hours. Excess phosgene and diluent are removed under reduced pressure. The crude product obtained as a residue is purified by distillation under reduced pressure.

184 g (91% of theory) of phthaloyl chloride of boiling point 123° C. (at 8 mbar) are obtained.

### Example 2

148 g (1.0 mol) of phthalic anhydride are dissolved in 250 ml of chlorobenzene and the mixture is heated to 75° C. At this temperature, after addition of 1.6 g of N,N-dibutylformamide (total amount: 11.2 g, 71 mmol) phosgene is introduced in a uniform stream, and about 28 g of phosgene are metered in within about 45 minutes in each case. After each period of about 45 minutes, 1.6 g of N,N-dibutylformamide are added to the mixture until the total amount has been consumed (phosgene: 198 g in total, 2.0 mol). Subsequently, the reaction mixture is stirred at 75° C. for a further 2 hours. The excess phosgene is then removed substantially by introducing nitrogen, and the diluent is distilled off under reduced pressure. The crude product obtained as the residue is purified by distillation under reduced pressure.

176 g (87% of theory) of phthaloyl chloride of boiling point 112° C. (at 4 mbar) are obtained.

### Example 3

148 g (1.0 mol) of phthalic anhydride are taken up in 250 ml of chlorobenzene and the mixture is heated to 75° C. At this temperature, after addition of 0.75 g of N,N-dimethylformamide (total amount: 5.2 g, 71 mmol) phosgene is introduced in a uniform stream, and about 28 g of phosgene are metered in within about 45 minutes in each case. After each period of about 45 minutes, 0.75 g of N,N-dimethylfonmamide are added to the mixture until the total amount has been consumed (phosgene: 198 g in total, 2.0 mol). Subsequently, the reaction mixture is stirred at 75° C. for a further 2 hours. The excess phosgene is then removed substantially by introducing nitrogen, and the diluent is distilled off under reduced pressure. The crude product obtained as the residue is purified by distillation under reduced pressure.

184 g (91% of theory) of phthaloyl chloride of boiling point 112° C. (at 4 mbar) are obtained.

1-5. (canceled)

6. A process for preparing phthaloyl chloride of formula (I) 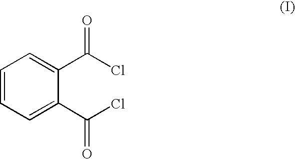
comprising reacting phthalic anhydride of formula (II) 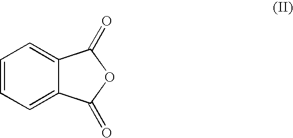
with phosgene in the presence of an N,N-dialkylformamide of formula (III) 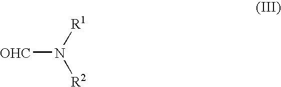
in which R¹ and R² are each independently straight-chain or branched alkyl, in the presence of an inert diluent at temperatures between 20° C. and 150° C., wherein the phosgene and the N,N-dialkylformamide of the formula (III) are independently metered in continuously or semicontinuously.

7. The process according to claim 6 wherein the N,N-dialkylformamide of formula (III) is metered in continuously.

8. The process according to claim 6 wherein the N,N-dialkylformamide of formula (III) is metered in semicontinuously.

9. The process according to claim 6 wherein the phosgene is metered in continuously.

10. The process according to claim 7 wherein the phosgene is metered in continuously.

11. The process according to claim 8 wherein the phosgene is metered in continuously.

12. The process according to claim 6 wherein the phosgene is metered in semicontinuously.

13. The process according to claim 7 wherein the phosgene is metered in semicontinuously.

14. The process according to claim 8 wherein the phosgene is metered in semicontinuously.
